(19) United States
(12) Patent Application Publication
(10) Pub. No.: US 2024/0390390 A1
(43) Pub. Date: Nov. 28, 2024

(54) NITRIC OXIDE RELEASING PROSTAMIDE AS NEUROPROTECTIVE AGENT

(71) Applicant: Nicox SA, Valbonne (FR)

(72) Inventors: Francesco IMPAGNATIELLO, Milano (IT); Elena BASTIA, Milano (IT)

(21) Appl. No.: 18/693,676

(22) PCT Filed: Sep. 20, 2021
(86) PCT No.: PCT/EP2021/075786

§ 371 Date: Mar. 20, 2024

(51) Int. Cl. A61K 31/5575; A61K 9/08; A61K 45/06; A61K 47/02; A61K 47/10; A61K 47/14; A61K 47/26; A61P 27/02

(52) U.S. Cl. CPC A61K 31/5575; A61K 9/08; A61K 45/06; A61K 47/02; A61K 47/10; A61K 47/14; A61K 47/26; A61P 27/02

(57) ABSTRACT

The present invention relates the compound hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) effective for treating normal tension glaucoma, neovascular glaucoma and ocular diseases associated with retinopathy.

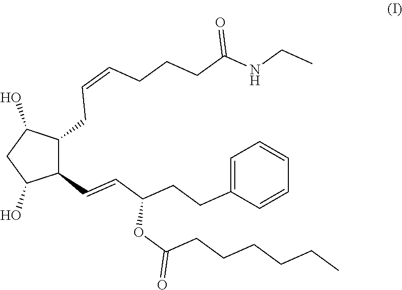 [SMILES: CCCCCCC(=O)O[C@H](/C=C/[C@@H]1[C@@H](C/C=C\CCCC(=O)NCC)[C@@H](O)C[C@H]1O)CCc1ccccc1]

### FIELD OF THE INVENTION

The present invention relates to the use of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) in a method for treating normal tension glaucoma (NTG), neovascular glaucoma (NVG) and in an adjuvant method of treatment of ocular diseases associated with retinopathy.

### BACKGROUND OF THE INVENTION

Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) has the following formula:

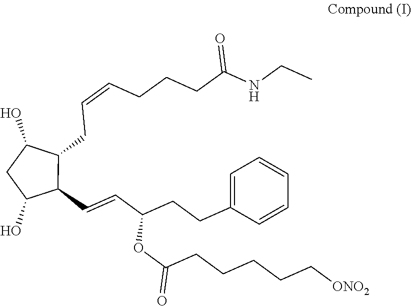

The efficacy of Compound (I) for the reduction of elevated intraocular pressure (IOP) was evaluated in a Phase 2 clinical trial conducted in 433 patients with open-angle glaucoma or ocular hypertension, treated once daily for 27 days. The results of the study demonstrated that Compound (I) at the concentration of 0.065% reduced IOP from baseline by 7.6 to 9.8 mmHg. In the same study, Compound (I) was well tolerated with the most frequent adverse event being conjunctival hyperemia occurring in 16.8% of treated patients with the highest dose tested.

EP 2 274 279 discloses the synthesis of Hexanoic acid, 6-(nitrooxy)-, (1S,2E)-3-[(1R,2R,3 S,5R)-2-[(2Z)-7-(ethylamino)-7-oxo-2-hepten-1-yl]-3,5-dihydroxycyclopentyl]-1-(2-phenylethyl)-2-propen-1-yl ester (Example B-1) and its use as IOP-reducing agent in patients with Primary Open Angle Glaucoma (POAG) or ocular hypertension (OHT).

Normal tension glaucoma is a type of progressive glaucomatous optic neuropathy in which damage occurs to the optic nerve without eye pressure exceeding the normal range; by definition, normal tension glaucoma differs from POAG in that the intraocular pressure is constantly below 21 mmHg (Kamal & Hitchings, British Journal Ophthalmology 1998; 82(7):835-40). Normal tension glaucoma is also referred to as low-tension glaucoma, normal-pressure glaucoma or normotensive glaucoma.

Normal tension glaucoma is characterized by glaucomatous optic neuropathy and progressive loss of visual field.

Several population-based studies have shown that, normal tension glaucoma occurs more frequently in Asian populations. Some studies suggest that, on average, 52-92% of all glaucoma cases are diagnosed as normotensive glaucoma, depending on the country (Killer & Pircher A. Normal tension glaucoma: review of current understanding and mechanisms of the pathogenesis. 2018; 32(5):924-930. Sheybani et al, Open-Angle Glaucoma: Burden of Illness, Current Therapies, and the Management of Nocturnal IOP Variation. Ophthalmol Ther. 2020; 9(1):1-14).

Although several clinical trials confirmed the value of reducing IOP in normal tension glaucoma, it is believed that the interaction of a variety of IOP-independent risk factors may be involved in the onset and progression of this disease. For example, damage to the optic nerve head in NTG is thought to be the result of abnormal retinal blood flow causing episodes of transient ischemic insult of the optic nerve (Choi & Kook, Systemic and Ocular Hemodynamic Risk Factors in Glaucoma. Biomed Res Int. 2015; 2015:141905; Drance Br. J. Oph. 1972; 56:229-242). Recently, elevated aqueous and plasma levels of endothelin-1 (ET-1) have been linked to glaucoma, in particular upregulation of ET-1 may be involved in the pathogenesis of NTG, where vascular dysregulation and other IOP-independent mechanisms seem to contribute (Choi & Kook, Systemic and Ocular Hemodynamic Risk Factors in Glaucoma. Biomed Res Int. 2015; 2015:141905).

IOP-lowering agents remain the only proven effective therapy for the treatment of NTG. The Collaborative Normal-Tension Glaucoma Study demonstrated that a 30% reduction in IOP decreased the long-term risk of visual field change from 35% to 12% (Am J Ophthalmol 1998; 126: 498-505).

Prostaglandin analogues are the mainstay IOP-lowering therapy; these drugs demonstrated to be the most effective IOP-lowering agents providing adequate diurnal control of IOP (Cheng et al., Meta-analysis of medical intervention for normal tension glaucoma. Ophthalmology 2009, 116: 1243-1249).

Another class of frequently prescribed IOP-lowering agents includes beta blockers. Prior to the introduction of prostaglandin analogues, topical beta blockers were considered as first-line IOP-lowering agents. However, topical beta blockers have the potential for significant systemic side effects, such as nocturnal systemic hypotension, that may be of particular concern in NTG. Dorzolamide (2%) and timolol (0.5%) fixed combination was reported as an effective IOP-lowering agent in patients with NTG (J Glaucoma 2014; 23:239).

The results of a study which compared the effects of brimonidine (0.2%) and timolol (0.5%) in monotherapy for NTG, showed that brimonidine-treated patients were statistically less likely to encounter progressive visual field loss than patients treated with timolol despite comparable IOP decreases. This finding suggested that brimonidine might have had additional, IOP independent neuroprotective effect that delayed the progressive visual field loss; however, more brimonidine-treated patients suffered adverse events, mainly ocular allergy (Am J Ophthalmol 2011; Vol. 151, No 4: 671-681).

Neovascular glaucoma is a sight-threatening secondary glaucoma characterized by the appearance of new vessels over the iris and proliferation of fibrovascular tissue in the anterior chamber angle. Retinal ischemia is also a common driving factor although several pathological conditions can ultimately lead to NVG, central retinal vein occlusion, proliferative diabetic retinopathy, and ocular ischemic syndrome are most common concurrent conditions that may ultimately lead to NVG.

Retinal hypoxia and ischemia are important factors in the pathogenesis of NVG. Acute painful eye, decreased vision, increased IOP, and dilation of circumciliary blood vessels are most common symptoms in NVG. While photocoagulation is the current treatment of choice wherever retinal ischemia is the cause of NVG, typical IOP-lowering medications including carbonic anhydrase inhibitors (oral and topical), beta-blockers, and alpha-2 agonists, or prostaglandin analogs are effective treatment in NVG patients especially when this condition is associated with an IOP increase. However, IOP-lowering agents alone can sometime aggravate concurrent ocular inflammation often observed in NVG patients.

Neuroprotective agents represent an emerging therapy for glaucoma, for example Unoprostone is a prostanoid that is approved for reduction of IOP in primary open angle glaucoma and ocular hypertension; this compound increases aqueous outflow via the trabecular meshwork and lowers IOP. Furthermore, preclinical studies suggested that unoprostone may also prolong neuronal survival independently from its ability to lower IOP; this effect seems in part due to improved ocular blood flow via inhibition of ET-1-induced phosphorylation of extracellular signal-regulated kinase (Munemasa et al., Visual Neuroscience 2008; 25:197-208). However, further studies are needed to confirm the neuroprotective properties of this compound prior to clinical evaluation.

Glutamate is an excitatory neurotransmitter in the CNS and retina, which is cytotoxic at high extracellular levels. Experimental models of glaucoma have shown glutamate excitotoxicity to be associated with retinal ganglion cell death and the inhibition of excess glutamate release or blockade of its receptor, N-methyl-D-aspartate (NMDA), has been proposed as a potential therapeutic target for neuroprotection in glaucoma (Guo et al., Invest Ophthalmol vis Sci 2006; 47: 626-633).

Furthermore, drugs that act on ocular blood flow have been tested, for example calcium channel blockers, such as nimodipine, normalized the retinal blood flow in NTG patients with vasospastic symptoms and increased choroidal blood flow. However, its benefit still needs validation in well-controlled randomized clinical trials.

Nitric Oxide donating prostaglandin derivatives have been studied as IOP-lowering compounds for the treatment of glaucoma or ocular hypertension. WO 2018/087092 discloses the use of nitrooxyalkyl ester of latanoprost acid (Latanoprostene Bunod) for the treatment of normal tension glaucoma. WO 2018/087092 discloses the results of a post-hoc analysis of two multicenter Phase 3 clinical studies in Caucasian subjects and of a multicenter Phase 3 clinical study in Japanese subjects; these studies evaluated the IOP-lowering effect of 0.024% Latanoprostene Bunod once daily in the evening for up to 12 months in subjects with open-angle glaucoma or ocular hypertension. Non-study (i.e. fellow) eyes with normal IOP (≤21 mmHg) at baseline in the Caucasian population and non-study (i.e. fellow) eyes with normal IOP at baseline (≤19 mmHg) in the Japanese population were identified and IOP outcomes were analyzed; the results showed that Latanoprostene Bunod lowered IOP from baseline by 4.2-4.9 mmHg and by 3.2-3.9 mmHg in the Caucasian and Japanese population, respectively (Fingeret American Academy of Optometry, 2016). Latanoprostene Bunod corresponds to Compound (1) of WO 2018/087092 and it has been marketed under the brand name Vyzulta® for the reduction of ocular hypertension and glaucoma. However, WO 2018/087092 does not disclose any neuroprotective effect on retina and/or optic nerve head related to treatment with nitrooxyalkyl esters of latanoprost acid.

Surgical intervention is the next step in cases of patients who fail to respond to the IOP reducing pharmacotherapy. Surgical management of normal tension glaucoma includes trabeculectomy, trabeculoplasty and, most recently, the “non-penetrating” glaucoma surgery, which includes selective laser visocanalostomy and deep sclerectomy, has gained interest for its potential to limit some of the complications associated with more invasive procedures to lower IOP.

Retinopathy or retinal vascular disease or retinal dysfunction is a degenerative condition of the retina that may lead to visual field loss or blindness and can be caused by various ocular as well as numerous systemic diseases. Ocular diseases such as diabetic macular edema (DME), retinopathy of prematurity (ROP), hypertensive retinopathy, Sickle cell retinopathy, age-related macular degeneration (AMD) and retinal vein occlusion (RVO) are characterized by retinal cell dysfunction.

Diabetic retinopathy results from damage to the retina consequent to diabetes mellitus. Retinopathy of prematurity is often observed in preterm neonates and involves insults that disrupt neurovascular growth in retina vessels; hypertensive retinopathy is due to high systemic blood pressure. In this disease, some blood vessels can narrow, thicken and harden with flame-shaped hemorrhages and macular swelling around the retina causing distorted or decreased vision. Sickle cell retinopathy is often observed in patients experiencing sickle cell anemia; these patients don't typically have visual symptoms early in the disease, however, retinal neovascularization likely occurs in late stages thereby causing vision loss.

Current pharmacological treatments for diabetic macular edema and other forms of retinopathies include anti-vascular endothelial growth factor (anti-VEGF) therapies such as intravitreal bevacizumab (Avastin®), ranibizumab (Lucentis®) and aflibercept (Eylea®), and steroids such as intravitreal triamcinolone acetonide, fluocinolone acetonide implantable device, and extended-release dexamethasone implant. Although anti-VEGF therapies and steroids are effective in improving visual acuity and the overall quality of life of patients, published reports also highlight the potential for a significant rise in intraocular pressure induced by these treatments (Sudhalkar et al., Current intravitreal therapy and ocular hypertension: A review. Indian J Ophthalmol. 2021; 69(2):236-243.; Razeghinejad & Katz Ophthalmic Res. 2012; 47(2):66-80).

### SUMMARY OF THE INVENTION

An object of the present invention is to provide an effective therapy to treat normal tension glaucoma (NTG), neovascular glaucoma (NVG) and retinopathy.

Accordingly, the present invention provides the compound Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) for use in a method for the treatment of normal tension glaucoma, neovascular glaucoma, and for use as adjuvant therapy for treating ocular diseases associated with retinopathy that are typically treated with primary therapies that elevate intraocular pressure as an undesired effect.

The inventors found that Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester independently of its demonstrated IOP lowering activity, effectively counteracts the hemodynamic changes induced by repeated endothelin-1 (ET-1) injection in rabbit eyes. Furthermore, Compound (I) also inhibited ET-1 changes on retinal cell physiology. These results suggest that Compound (I) targets IOP-independent risk factors such as retinal cell viability, vascular dysfunction and optic nerve head damage all known to participate in the progression of normal tension glaucoma, neovascular glaucoma or other ocular diseases associated with retinopathy.

The advantages of the use of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester in the therapeutic method of the invention are the following: Compound (I) combines IOP-lowering dependent effects and IOP-lowering independent retinal and optic nerve neuroprotective effects; both effects resulting protective against visual disability or blindness in patients with normal tension glaucoma or with neovascular glaucoma. The vascular protective effect of Compound (I) ameliorates ocular perfusion, making Compound (I) suitable to be used as adjuvant treatment in the pharmacological management of retinal dysfunction, associated with ocular diseases, in addition, the IOP-lowering efficacy of Compound (I) counteracts the adverse elevation of IOP frequently observed by treatments with intravitreal anti-VEGF agents or with steroids.

Ocular diseases associated with retinopathy that can be treated using the method of the invention include, but are not limited to, diabetic macular edema (DME), retinopathy of prematurity (ROP), hypertensive retinopathy, Sickle Cell retinopathy, retinal vein occlusion (RVO) and age-related macular degeneration (AMD).

An embodiment of the invention provides Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester (Compound (I)) for the use in the treatment of normotensive glaucoma or neovascular glaucoma.

Another embodiment of the invention provides Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) for the use in a method of treating an ocular disease in a subject receiving a primary pharmacological treatment with intravitreal anti-vascular endothelial growth factor (anti-VEGF) agent or with a steroid, wherein said Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester is used as an adjuvant therapy and wherein the ocular disease is selected from diabetic macular edema, retinopathy of prematurity, hypertensive retinopathy, Sickle cell retinopathy, retinal vein occlusion or age-related macular degeneration; preferably the ocular disease is diabetic macular edema or retinal vein occlusion; preferably the anti-vascular endothelial growth factor agent is selected from: bevacizumab (Avastin®), ranibizumab (Lucentis®) or aflibercept (Eylea®); preferably the steroid is selected from: triamcinolone acetonide, fluocinolone acetonide implant, or extended-release dexamethasone implant.

In an embodiment of the present invention the adjuvant therapy with Compound (I) comprises administering Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) during and between treatments with the primary pharmacological agent with the anti-VEGF agent or with the steroid. The administration includes administering Compound (I) once a day or twice a day before and during intervals between primary pharmacological treatment consecutive dosing.

The amounts of Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester is administered depend on the judgment of the practitioner and are peculiar to each individual. Suitable dosages may range from about 3 μg/eye/dose to 300 μg/eye/dose; preferably from about 9 μg/eye/dose to about 90 μg/eye/dose; more preferably from 30 μg/eye/dose to about 60 μg/eye/dose.

Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester is administered as an ophthalmic formulation, a preferred ophthalmic formulation is eye drops. Such eye drops may be aqueous eye drops, non-aqueous eye drops, emulsion eye drops, ophthalmic ointment, and the like; ophthalmic aqueous eye drop solutions are preferred.

The eye drops comprise Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester and further comprise one or more conventional excipients used in ophthalmic compositions selected from: isotonic agents, chelating agents, stabilizers, solubilizers, surfactants, viscosity enhancing agents, buffers, pH adjusting agents, antimicrobial preservative agents or antioxidants. The pH of the ophthalmic solution according to the invention is preferably from 4 to 8, more preferably from 5 to 7.

An embodiment of the invention provides Hexanoic acid, 6-(nitrooxy)-, (1S,2E)-3-[(1R,2R,3 S,5R)-2-[(2Z)-7-(ethylamino)-7-oxo-2-hepten-1-yl]-3,5-dihydroxy cyclopentyl]-1-(2-phenylethyl)-2-propen-1-yl ester (Compound (I)) for use in the treatment of normotensive glaucoma or neovascular glaucoma wherein said Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester is administered as an eye drop aqueous solution comprising 0.01% to 0.18% w/w hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester, 0.5% w/w to 1.5% w/w macrogol 15 hydroxystearate, a buffer, a tonicity agent and water, and having a pH in the range from 5.5 to 6.5 and an osmolality from 260 to 340 mOsm/Kg; preferably the amount of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester in the ophthalmic aqueous solution is 0.021% w/w, 0.042% w/w, 0.065% w/w or 0.1% w/w; optionally the ophthalmic aqueous solution further comprises benzalkonium chloride and ethylenediaminetetraacetic acid disodium salt (EDTA).

Preferably the tonicity agent is sorbitol or glycerol.

Another embodiment of the invention provides Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester (Compound (I)) for use in the treatment of normotensive glaucoma or neovascular glaucoma, wherein said Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester is administered as an eye drop aqueous solution comprising 0.01% to 0.18% w/w hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester, 0.5% w/w to 1.5% w/w macrogol 15 hydroxystearate, sorbitol or glycerol, sodium phosphate dibasic heptahydrate, boric acid, and water, and having a pH in the range from 5.5 to 6.5 and an osmolality from 260 to 340 mOsm/Kg; preferably the aqueous solution contains 0.021% w/w, 0.042% w/w, 0.065% w/w or 0.1% w/w of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester; optionally the ophthalmic aqueous solution further comprises benzalkonium chloride and ethylenediaminetetraacetic acid disodium salt (EDTA).

Another embodiment of the invention provides Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) for use in a method of treating an ocular disease in a subject receiving a primary pharmacological treatment with an intravitreal anti-vascular endothelial growth factor agent or with a steroid, wherein said Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester is administered as adjuvant therapy and wherein said Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester is administered as an eye drop aqueous solution comprising 0.01% to 0.18% w/w hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester, 0.5% w/w to 1.5% w/w macrogol 15 hydroxystearate, a tonicity agent, a buffer, water and having a pH in the range from 5.5 to 6.5 and an osmolality from 260 to 340 mOsm/Kg and wherein the ocular disease is selected from diabetic macular edema, retinopathy of prematurity, hypertensive retinopathy, Sickle cell retinopathy, retinal vein occlusion or age-related macular degeneration; preferably the ocular disease is diabetic macular edema or retinal vein occlusion; preferably the ophthalmic aqueous solution contains 0.021% w/w, 0.042% w/w, 0.065% w/w or 0.1% w/w of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester; optionally the ophthalmic aqueous solution further comprises benzalkonium chloride and ethylenediaminetetraacetic acid disodium salt (EDTA).

Another embodiment of the invention provides the use of Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) for treating an ocular disease in a subject receiving a primary pharmacological treatment with an intravitreal vascular endothelial growth factor agent or with a steroid, wherein said Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester is administered as adjuvant therapy and wherein said Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester is administered as an eye drop aqueous solution comprising 0.01% to 0.18% w/w hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester, 0.5% w/w to 1.5% w/w macrogol 15 hydroxystearate, sorbitol or glycerol, sodium phosphate dibasic heptahydrate, boric acid, and water and having a pH in the range from 5.5 to 6.5 and an osmolality from 260 to 340 mOsm/Kg and wherein the ocular disease is selected from diabetic macular edema, retinopathy of prematurity, hypertensive retinopathy, Sickle cell retinopathy, retinal vein occlusion or age-related macular degeneration; preferably the ocular disease is diabetic macular edema or retinal vein occlusion; preferably the aqueous solution contains 0.021% w/w, 0.042% w/w, 0.065% w/w or 0.1% w/w of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester; optionally the ophthalmic aqueous solution further comprises benzalkonium chloride and ethylenediaminetetraacetic acid disodium salt (EDTA).

As used herein, the term “adjuvant therapy”, “adjuvant treatment” or “adjuvant method of treatment” refers to a treatment given in addition to a primary pharmacological treatment.

As used herein, the term “primary pharmacological treatment” refers to therapies specifically used to treat the above mentioned ocular retinopathies that may also induce IOP elevation. Such “primary pharmacological treatment” includes anti-vascular endothelial growth factor agents (anti-VEGF) such as bevacizumab (Avastin®), ranibizumab (Lucentis®) and aflibercept (Eylea©), or the therapy with steroids such as triamcinolone acetonide, fluocinolone acetonide implant, extended-release dexamethasone implant.

### EXAMPLE 1

This series of experiments were performed to determine the effect of Compound (I) on ocular vascular reactivity i.e. ophthalmic artery resistive index (OA-RI) and, consequently, on retinal function after repeated ocular topical dosing in a well-defined model of ischemia/reperfusion injury of the optic nerve induced by the retrobulbar injection of endothelin-1 (ET-1) in rabbits.

### Test Items

Compound (I) dissolved in the vehicle (Cpd (I)) and the vehicle were tested in the study.

Cpd (I): 0.1% solution of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Compound (I)) dissolved in the vehicle.

Vehicle: 1.0% w/w macrogol 15 hydroxystearate, 0.016% w/w benzalkonium chloride, 0.05% w/w ethylenediaminetetraacetic acid disodium salt dihydrate, 2.76% w/w sorbitol, 1.33% w/w sodium phosphate dibasic heptahydrate, 0.5% w/w boric acid, water to 100% w/w and pH from 5.5 to 6.5.

Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester was synthetized according to the process disclosed in WO 2019/162149.

The 0.1% solution of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester (Cpd (I)) was prepared according to the method disclosed in WO 2020/011845.

### Method and Treatment

Adult male New Zealand white (NZW) rabbits weighting 1.5-2.0 kg were used. The animals were kept in individual cages, food and water were provided ad libitum. The ischaemia/reperfusion lesion of the optic nerve was obtained by subtenonian injection twice a week with 500 μL of 250 nM ET-1 (Fluka, Israel) in water using a lacrimal cannula under anesthesia produced by ketamine (20 mg/kg, Lobotor, ACME, 100 mg/ml) and xilazine (5 mg/kg, Sedaxylan 2%, Dechra Veterinary Products S.r.l) intramuscularly. Animals were treated with ET-1 for 6 weeks. Compound (I) 0.1% (1 mg/mL) solution or vehicle were administered as eye drop (30 μL/eye), twice a day (around 10:00 AM and 4:00 PM) starting 2 weeks after ET-1 first dosing until the end of the experiment.

Ophthalmic Artery Resistive Index (OA-RI) was estimated using an ecocolor Doppler DynaView™ II SSD-1700 (Aloka Holding Europe AG, Milan, Italy) prior to ET-1 treatment (baseline, time 0), and weekly thereafter until the end of the study. From week 2 to week 4, morning dosing with test article or vehicle were omitted on the day of hemodynamic measurements in order to have virtually drug-free conditions (16-18 h post last dose) at the time of testing. Pourcelot resistive index for ophthalmic artery (OA-RI) was calculated as following: (PSV−EDV)/PSV where PSV and EDV refer to Peak Systolic Velocity and End Diastolic Velocity, respectively.

Electroretinogram (ERG). Topical anesthesia was applied using one drop 0.2% oxybuprocaine hydrochloride, 4 mg/ml. The eyes were then dilated by topical application of tropicamide 1% and, when needed, adapted to darkness for at least 2 hours prior to standard ERGs recording in both eyes using contact lens corneal electrodes so to have sufficiently stable and amplified recordings. The ERG signals [(amplitude, microvolts (μV)] were recorded using Retimax (CSO, Florence, Italy) and according to the current International Society for Clinical Electrophysiology (ISCEV) indications; specifically, the dark-adapted 0.01 ERG (rod response), dark-adapted 3.0 ERG (combined rod-cone response) and, light-adapted 3.0 (cone response) were recorded. Flashes intensity varied:
a) from 0.01 photopic cd·s·m⁻² to 0.025 scotopic candela-seconds per meter squared (cd·s·m⁻²) with a minimum interval between flashes of 2 s for the dark-adapted 0.01 ERG;
b) from 3.0 photopic cd·s·m⁻² to 7.5 scotopic cd·s·m⁻² with a minimum interval between flashes of 10 s for the dark-adopted 3.0 ERG
c) from 3.0 photopic cd·s·m⁻² to 7.5 scotopic cd·s·m⁻² with a minimum interval between flashes of 0.5 s and a light adaptation strength of 30 cd·s·m⁻² for the light-adopted 3.0 ERG. In all cases ERG recordings lasted 250 ms. ERG recordings lasted 250 ms.

Measurements were taken prior ET-1 first dose (baseline, time 0) and then at the end of week-2 (prior to vehicle- or Compound (I) first day-first dose) and at the end of week-6 (36 h after vehicle or Compound (I)-last day-last dose).

### Results

### Baseline Ophthalmic Artery Resistive Index (OA-RI)

Prior to endothelin-1 (ET-1) dosing, OA-RI values were 0.30±0.02 and 0.30±0.02 respectively in animals randomized to vehicle or Compound (I) treatments (Table 1). Twice weekly dosing with ET-1 for 2 weeks elevated OA-RI. In the animals treated with vehicle, OA-RI continued increasing over the following 4 weeks. In animals treated with Compound (I) OA-RI tend to decrease (0.32±0.03 and 0.33±0.02 on week 5 and 6, respectively) (Table 1) likely as a result of the compensatory effect of Compound (I) on the effects of ET-1.

TABLE 1
Ophthalmic artery resistive index (OA-RI) values at different time-points

| | | Dosing schedule | | | | |
|---|---|---|---|---|---|---|
| | | | ET-1 | ET-1 | ET-1 | ET-1 |
| | | | (3-weeks) + | (4-weeks) + | (5 weeks) + | (6 weeks) + |
| | | | Test items | Test items | Test items | Test items |
| Test | | ET-1 | (1 week | (2 weeks | (3 weeks | (4 weeks |
| items | Baseline | (2 weeks) | daily bid) | daily bid) | daily bid) | daily bid) |
| Vehicle | 0.30 ± 0.02 | 0.39 ± 0.02 | 0.40 ± 0.03 | 0.41 ± 0.02 | 0.42 ± 0.02 | 0.42 ± 0.03 |
| Cpd (1) | 0.30 ± 0.02 | 0.36 ± 0.03 | 0.35 ± 0.03 | 0.32 ± 0.03* | 0.32 ± 0.03* | 0.33 ± 0.02* |

*P < 0.05 vs vehicle

### Electroretinogram (ERG)

The Electroretinogram (ERG) responses are reported in Table 2. ET-1 treatment resulted in a marked decline in retinal functions two weeks after the injection of ET-1 and continued to decrease thereafter as shown by the low amplitude recorded at week 6 in eyes treated with ET-1. However, eyes treated for 4 weeks with Compound (I) exhibited significantly (p<0.05) less impairment in the ERG wave amplitude than those treated with vehicle regardless of the stimuli (dark-adapted 0.01, dark-adapted 3.0 and Light-adapted 3.0) applied.

TABLE 2
Electroretinogram (ERG) responses at different time-points

| | | ERG Amplitude (μV ± S.E.M) | | |
|---|---|---|---|---|
| | | | | ET-1 |
| | | | | (6 weeks) |
| | | | | + |
| | | | | Test items |
| Test | | | ET-1 | (4 weeks |
| items | ERG stimuli | Baseline | (2 weeks) | daily bid) |
| Vehicle | Dark adapted 0.01 | 49.3 ± 4.7 | 38.0 ± 3.4 | 32.2 ± 3.0 |
| | (Rod response) | | | |
| | Dark adapted 3.0 | 109.8 ± 12.9 | 98.6 ± 6.6 | 87.6 ± 10.1 |
| | (Rod/cone response) | | | |
| | Light adapted 3.0 | 70.4 ± 8.8 | 59.9 ± 2.9 | 49.8 ± 6.5 |
| | (cone response) | | | |
| Cpd (1) | Dark adapted 0.01 | 42.3 ± 6.9 | 37.6 ± 6.2 | 44.3 ± 4.5* |
| | (Rod response) | | | |
| | Dark adapted 3.0 | 118.0 ± 10.3 | 96.6 ± 9.4 | 122.8 ± 11.4* |
| | (Rod/cone response) | | | |
| | Light adapted 3.0 | 67.6 ± 6.4 | 60.2 ± 4.3 | 64.2 ± 6.8 |
| | (cone response) | | | |

*P < 0.05 vs vehicle

In summary, the results showed that hexanoic acid, 6-(nitrooxy)-, (1S,2E)-3-[(1R,2R,3 S, 5R)-2-[(2Z)-7-(ethylamino)-7-oxo-2-hepten-1-yl]-3,5-dihydroxycyclopentyl]-1-(2-phenylethyl)-2-propen-1-yl ester ameliorated ocular vascular reactivity i.e. ophthalmic artery resistive index (GA-RI) and retinal function i.e. ERG after repeated ocular topical dosing in a well-defined model of ischemia/reperfusion injury of the optic nerve induced by the retrobulbar injection of endothelin-1 in rabbits.

These data provide evidence of an improved ocular perfusion and retinal cell physiology after Compound (I) dosing which may ultimately lead to reverse optic nerve degeneration and retina cell dysfunction consequent to repeated ischemic lesions.

These results support the use of Hexanoic acid, 6-(nitrooxy)-, (1S,2E)-3-[(1R,2R,3 S, 5R)-2-[(2Z)-7-(ethylamino)-7-oxo-2-hepten-1-yl]-3,5-dihydroxycyclopentyl]-1-(2-phenylethyl)-2-propen-1-yl ester in the treatment of ocular pathologies in which retinal neuroprotection secondary to re-established ocular perfusion is a key factor to halt the progression of the diseases.

1. Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester for use for the treatment of normotensive glaucoma or neovascular glaucoma.

2. Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester for use according to claim 1 wherein said Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester is administered as an ophthalmic formulation.

3. Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester for use according to claim 2 wherein the ophthalmic formulation is an eye drop aqueous solution comprising 0.01% to 0.18% w/w hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester, 0.5% w/w to 1.5% w/w macrogol 15 hydroxystearate, a tonicity agent, a buffer, water and having a pH in the range from 5.5 to 6.5 and an osmolality from 260 to 340 mOsm/Kg.

4. Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester for use according to claim 3 wherein the tonicity agent is sorbitol or glycerol.

5. Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester for use according to claim 2 wherein the ophthalmic formulation is an eye drop aqueous solution comprising 0.01% to 0.18% w/w hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxy cyclopentyl]-1-(2-phenylethyl)-2-propen-1-yl ester, 0.5% w/w to 1.5% w/w macrogol 15 hydroxystearate, sorbitol or glycerol, sodium phosphate dibasic heptahydrate, boric acid, and water, and having a pH in the range from 5.5 to 6.5 and an osmolality from 260 to 340 mOsm/Kg.

6. Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester for use according to claim 2 wherein the eye drop aqueous solution contains 0.021% w/w, 0.042% w/w, 0.065% w/w or 0.1% w/w of hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester.

7. Hexanoic acid, 6-(nitrooxy)-, (1S,2E)-3-[(1R,2R,3S,5R)-2-[(2Z)-7-(ethylamino)-7-oxo-2-hepten-1-yl]-3,5-dihydroxycyclopentyl]-1-(2-phenylethyl)-2-propen-1-yl ester for use according to claim 3 wherein the eye drops aqueous solution further comprises benzalkonium chloride and ethylenediaminetetraacetic acid.
